(19) United States
(12) Patent Application Publication
(10) Pub. No.: US 2026/0020921 A1
(43) Pub. Date: Jan. 22, 2026

(54) SURGICAL SPINOUS PROCESS CLAMP

(71) Applicant: Medivis, Inc., New York, NY (US)

(72) Inventors: Jesse Harrison Gallant, New York, NY (US); Osamah Choudhry, New York, NY (US); Christopher Morley, New York, NY (US); Florentin Jonas Liebmann, New York, NY (US)

(21) Appl. No.: 19/344,152

(22) Filed: Sep. 29, 2025

Related U.S. Application Data
(63) Continuation-in-part of application No. 18/135,663, filed on Apr. 17, 2023.
(60) Provisional application No. 63/700,748, filed on Sep. 29, 2024.
(60) Provisional application No. 63/700,747, filed on Sep. 29, 2024.

(51) Int. Cl. A61B 34/20; A61B 90/00

(52) U.S. Cl. CPC A61B 34/20; A61B 2034/2072; A61B 2090/3916; A61B 2090/3991

(57) ABSTRACT

A spinous process clamp includes a main body, first arm, second arm, shaft, traveling component, and clamp coupling arrangement. The main body has a top region, first and second side regions and a pin extending therebetween. Each arm has a top portion, bottom portion, and midsection configured to rotate about the pin. The shaft extends through the top region of the main body. The traveling component is configured to travel along the shaft and to push against the top portions of the first and second arms when the traveling component travels sufficiently downward along the shaft. Pushing against the arm top portions causes the arms to rotate about the pin such that the arm bottom portions clamp onto a spinous process placed therebetween. The clamp coupling arrangement couples the spinous process clamp to a separate surgical device, such as a fiducial marker array, and the coupling can be magnetic.

### CROSS REFERENCE TO RELATED APPLICATIONS

This application claims the benefit of priority to U.S. Provisional Application Nos. 63/700,748, filed Sep. 29, 2024, and 63/700,747, filed Sep. 29, 2024, which are hereby incorporated by reference in their entirety. This application is also a continuation in-part of U.S. application Ser. No. 18/135,663, filed on Apr. 17, 2023, which is hereby incorporated by reference in its entirety. This application is also related to commonly owned U.S. patent application Ser. No. 18/135,674, filed Apr. 17, 2023, and Ser. No. 18/900,106, filed Sep. 27, 2024, all applications of which are hereby incorporated by reference in their entireties herein.

### TECHNICAL FIELD

The present disclosure relates generally to medical devices, and more particularly to surgical tools and devices used during medical surgeries and procedures.

### BACKGROUND

Planning and navigation are necessary for many medical procedures, and surgical teams typically have a plan based on medical imagery before ever entering an operating room. Conventional medical imaging systems such as X-ray, MRI, CT, and others have limitations regarding two-dimensional and three-dimensional images, however, and surgeons often need to consider numerous image views and slices to plan surgical procedures. Recent medical advances leverage these applications of medical imagery and surgical plans by using a computer-aided augmented reality environment, which can allow for the tracking of patients and physical instruments during surgical procedures by using fiducial markers and tracking components.

Unfortunately, conventional tracking systems are often limited in their ability to accurately generate, render, and apply virtual interactions in an augmented reality environment based on the orientations and positions of physical instruments with respect to those of physical landmarks identified on a patient body, particularly when things move during surgery. Unstable or unreliable positioning of fiducial markers can play a role in these issues. Limited or inaccurate tracking can then affect the overall performance of such systems during surgical procedures, and the need for accuracy in this regard can lead to overly cumbersome or complex attachment devices and systems.

While traditional ways of virtually tracking items during surgery have worked well in the past, improvements are always helpful. In particular, what is desired are medical systems and devices that facilitate the stable and reliable positioning of fiducial markers during surgery in a simple and streamlined manner.

### SUMMARY

It is an advantage of the present disclosure to provide medical systems and devices that facilitate the stable and reliable positioning of fiducial markers during surgery in a simple and streamlined manner. The disclosed features, apparatuses, systems, and methods relate to clamps that can be used to affix surgical devices and equipment to patients during surgeries and other medical procedures. In particular, the disclosed systems and methods can involve surgical spinous process clamps that can be clamped onto a spinous process of a patient and that can be reliably coupled to one or more other surgical devices or items, such as a magnetically couplable fiducial marker array.

In various embodiments of the present disclosure, a surgical spinous process clamp can include a main body, first arm, second arm, shaft, traveling component, and clamp coupling component. The main body can have a top region, a first side region, a second side region opposite the first side region, and a pin extending between the first and second side regions. The first arm can have a top portion, a bottom portion, and a midsection configured to rotate about the pin in a first rotational direction. The second arm can be positioned opposite the first arm and can have a top portion, a bottom portion, and a midsection configured to rotate about the pin in a second rotational direction opposite the first rotational direction. The shaft can extend through the top region of the main body. The traveling component can be coupled to the shaft beneath the top region and configured to travel along the shaft and to push against the top portions of the first and second arms when the traveling component travels sufficiently downward along the shaft. Pushing against the top portions of the first and second arms can cause the first and second arms to rotate about the pin such that the arm bottom portions clamp onto a spinous process placed therebetween. The clamp coupling arrangement can be coupled to the main body and can be configured to couple the spinous process clamp to a separate surgical device.

In various detailed embodiments, the spinous process clamp can also include a first gripping component located proximate the bottom portion of the first arm and configured to grip a first side of the spinous process a second gripping component located proximate the bottom portion of the second arm and configured to grip a side of the spinous process opposite the first side. The separate surgical device can be a fiducial marker array. The clamp coupling arrangement can include one or more clamp magnetic components configured to form a magnetic coupling to one or more separate magnetic components of the separate surgical device. The magnetic coupling can result in the separate surgical device being set at a constant orientation and location with respect to the spinous process clamp while the separate surgical device remains magnetically coupled to the spinous process clamp. The shaft can include a threaded portion and the traveling component can be configured to interact with the threaded portion. The spinous process clamp can also include a spring coupled to the top portion of the first arm and the top portion of the second arm. The spring can bias the first and second arm top portions together to open the spinous process clamp at the first and second arm bottom portions by default. The main body can include a reference divot located on a surface thereof, the reference divot being configured to facilitate calibration of another separate surgical device having a pointer tip. The shaft can include a rotational control feature at the top region of the main body.

In various further embodiments of the present disclosure, various methods of using a spinous process clamp are provided. Pertinent process steps can include opening a distance between bottom portions of first and second arms of the spinous process clamp, placing the first and second arm bottom portions around a spinous process, forcing a traveling component down a shaft of the spinous process clamp so that the traveling component pushes against top portions of the first and second arms, and closing the arm bottom portions onto the spinous process as the traveling component pushes against the arm top portions.

In various detailed embodiments, further process steps can include receiving a separate surgical device into the clamp coupling arrangement to couple the separate surgical device to the spinous process clamp, and holding the separate surgical device set at a constant orientation and location with respect to the spinous process clamp while the separate surgical device remains coupled to the spinous process clamp. The coupling between the separate surgical device and the spinous process clamp can be a magnetic coupling.

Other apparatuses, methods, features, and advantages of the disclosure will be or will become apparent to one with skill in the art upon examination of the following figures and detailed description. It is intended that all such additional apparatuses, methods, features and advantages be included within this description, be within the scope of the disclosure, and be protected by the accompanying claims.

### BRIEF DESCRIPTION OF THE DRAWINGS

The included drawings are for illustrative purposes and serve only to provide examples of possible structures, arrangements, and methods of use for surgical spinous process clamps. These drawings in no way limit any changes in form and detail that may be made to the disclosure by one skilled in the art without departing from the spirit and scope of the disclosure.

FIG. 1A illustrates in front perspective view an example surgical fiducial marker system having a spinous process clamp according to one embodiment of the present disclosure.

FIG. 1B illustrates in side perspective view the surgical fiducial marker system of FIG. 1A according to one embodiment of the present disclosure.

FIG. 2A illustrates in front perspective view an example surgical fiducial marker system having a spinous process clamp according to one embodiment of the present disclosure.

FIG. 2B illustrates in front perspective view an example surgical fiducial marker system with a spinous process clamp according to another embodiment of the present disclosure.

FIG. 3A illustrates in front elevation view an example spinous process clamp at an open position according to one embodiment of the present disclosure.

FIG. 3B illustrates in front elevation view the spinous process clamp of FIG. 3A at a closed position according to one embodiment of the present disclosure.

FIG. 4 illustrates a flowchart of an example summary method of attaching a spinous process clamp to a spinous process according to one embodiment of the present disclosure.

FIG. 5A illustrates in front elevation view an example pair of gripping components coupled to spinous process clamp arms according to one embodiment of the present disclosure.

FIG. 5B illustrates in front elevation view the pair of gripping components of FIG. 5A at an alternative position according to one embodiment of the present disclosure.

FIG. 6A illustrates in front perspective view an example gripping component for a spinous process clamp according to one embodiment of the present disclosure.

FIG. 6B illustrates in rear perspective view the gripping component of FIG. 6A according to one embodiment of the present disclosure.

FIG. 6C illustrates in side elevation view the gripping component of FIG. 6A according to one embodiment of the present disclosure.

FIG. 7A illustrates in front elevation view an example pair of arms for a spinous process clamp according to one embodiment of the present disclosure.

FIG. 7B illustrates in front elevation view an example coupling arrangement for the pair of arms of FIG. 7A according to one embodiment of the present disclosure.

FIG. 8A illustrates in front perspective view an example adjustable spacing arrangement for a spinous process clamp according to one embodiment of the present disclosure.

FIG. 8B illustrates in side perspective view the adjustable spacing arrangement of FIG. 8A according to one embodiment of the present disclosure.

FIG. 9 illustrates in front perspective view an example receiving bearing for a spinous process clamp according to one embodiment of the present disclosure.

FIG. 10A illustrates in side perspective view an example clamp coupling component for a spinous process clamp according to one embodiment of the present disclosure.

FIG. 10B illustrates in top perspective view the clamp coupling component of FIG. 10A rotationally coupled to a positioner coupling component for a surgical fiducial marker positioner according to one embodiment of the present disclosure.

FIG. 11 illustrates a flowchart of an example detailed method of using a spinous process clamp according to one embodiment of the present disclosure.

FIG. 12A illustrates in front perspective view an example alternative surgical fiducial marker system having a surgical spinous process clamp and a magnetically couplable fiducial marker array according to one embodiment of the present disclosure.

FIG. 12B illustrates in top plan view the alternative surgical fiducial marker system of FIG. 12A according to one embodiment of the present disclosure.

FIG. 13A illustrates in front perspective view an example surgical spinous process clamp in an open position according to one embodiment of the present disclosure.

FIG. 13B illustrates in front perspective view an example surgical spinous process clamp in a closed position according to one embodiment of the present disclosure.

FIG. 14 illustrates in front perspective view a non-pivoting gripping component according to one embodiment of the present disclosure.

FIG. 15A illustrates in side cross-section view an example surgical spinous process clamp in an open position according to one embodiment of the present disclosure.

FIG. 15B illustrates in side cross-section view an example surgical spinous process clamp in a partially closed position according to one embodiment of the present disclosure.

FIG. 15C illustrates in side cross-section view an example surgical spinous process clamp in a fully closed position according to one embodiment of the present disclosure.

FIG. 16A illustrates in side perspective view an example surgical spinous process clamp with its main body removed in an open position according to one embodiment of the present disclosure.

FIG. 16B illustrates in side perspective view an example surgical spinous process clamp with its main body removed in a closed position according to one embodiment of the present disclosure.

FIG. 17A illustrates in top perspective view an example clamp magnetic coupling component of a surgical spinous process clamp according to one embodiment of the present disclosure.

FIG. 17B illustrates in bottom perspective view an example clamp magnetic coupling component of a surgical spinous process clamp according to one embodiment of the present disclosure.

FIG. 18 illustrates a flowchart of an example method of using a surgical spinous process clamp according to one embodiment of the present disclosure.

### DETAILED DESCRIPTION

Exemplary applications of apparatuses, systems, and methods according to the present disclosure are described in this section. These examples are being provided solely to add context and aid in the understanding of the disclosure. It will thus be apparent to one skilled in the art that the present disclosure may be practiced without some or all of these specific details provided herein. In some instances, well known process steps have not been described in detail in order to avoid unnecessarily obscuring the present disclosure. Other applications are possible, such that the following examples should not be taken as limiting. In the following detailed description, references are made to the accompanying drawings, which form a part of the description and in which are shown, by way of illustration, specific embodiments of the present disclosure. Although these embodiments are described in sufficient detail to enable one skilled in the art to practice the disclosure, it is understood that these examples are not limiting, such that other embodiments may be used, and changes may be made without departing from the spirit and scope of the disclosure.

As is generally well known, modern surgical plans and procedures are sometimes facilitated by using a computer-aided augmented reality environment. Surgical fiducial markers can be used for tracking of patients and physical instruments during surgical procedures in overall systems that can also include specialized lighting arrangements, cameras, and computing systems. Attachment devices are often used to affix the surgical fiducial markers in place relative to the patient, and unstable or unreliable positioning of these markers can result in the reduced effectiveness of the overall computer-aided augmented reality environment. The disclosed surgical attachment devices are specifically designed to facilitate the stable and reliable positioning of fiducial markers during surgery in a simple and streamlined manner.

The present disclosure relates in various embodiments to features, apparatuses, systems, and methods of use for surgical attachment devices, and in particular spinous process clamps. This can generally involve clamps or other suitable attachment devices that are configured to attach to patient bodies during surgical processes while also providing for the ability to be coupled to other surgical devices or equipment. In specific arrangements this can involve a spinous process clamp configured to clamp or attach to a spinous process while also having a surgical fiducial marker positioner attached thereto.

In various embodiments of the present disclosure, novel spinous process clamps can include gripping components located at or proximate the ends of opposing arms and configured to move with respect to the opposing arms. The opposing arms can be configured to move such that the gripping components can be closed to clamp the spinous process clamp onto a spinous process and can be opened to remove the spinous process clamp from the spinous process. An adjustable spacing arrangement can be coupled to both arms and can be configured to enable controlled arm movements that open and close the distance between the gripping components. A coupling component coupled to at least one arm can be configured for coupling the spinous process clamp to a separate surgical component, such as a surgical fiducial marker positioner. In some arrangements, the adjustable spacing arrangement and the coupling component can be located at or proximate arm ends opposite the ends with the gripping components.

Although various embodiments disclosed herein discuss the use of a spinous process clamp in conjunction with a surgical fiducial marker positioner as part of an overall surgical fiducial marker system, it will be readily appreciated that the disclosed features, apparatuses, systems, and methods can also be used in conjunction with other devices and equipment that can leverage the advantages of such a clamp. While the disclosed spinous process clamps are contemplated for use involving clamping to spinous processes during a surgical procedure, it is specifically contemplated that other clamped objects and applications may also be applied. For example, the disclosed spinous process clamps can be used to clamp to other bones or body parts for surgical and non-surgical uses, such as during examination and testing procedures. Further, other devices and equipment that can be coupled to the disclosed spinous process clamps can include lighting arrangements, sensing devices, or other desired equipment. Other applications, arrangements, and extrapolations beyond the illustrated embodiments are also contemplated.

### Spinous Process Clamps

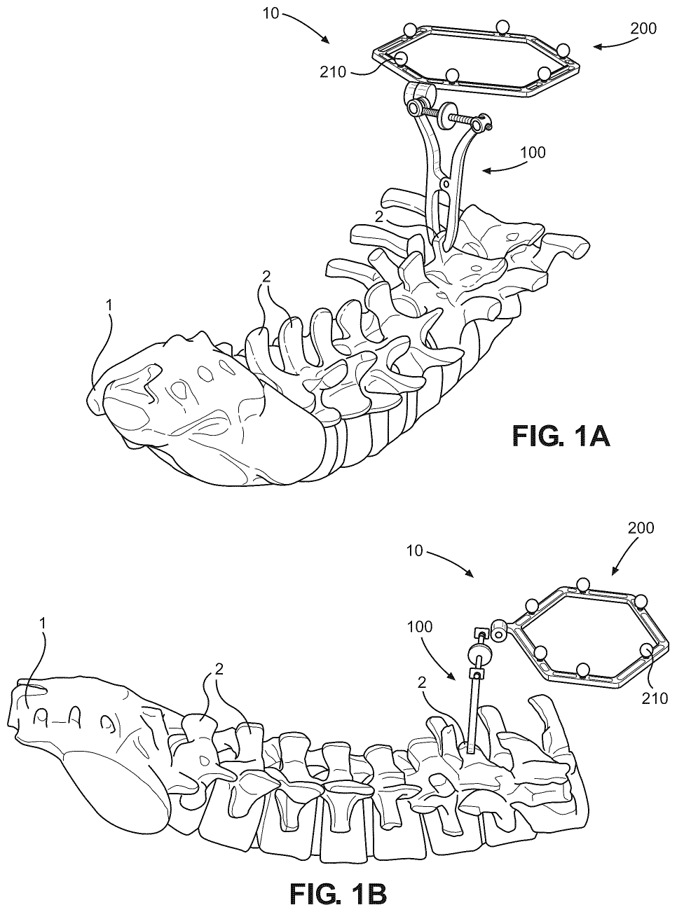

Referring first to FIGS. 1A and 1B, an example surgical fiducial marker system having a spinous process clamp is illustrated in front perspective and side perspective views respectively. Surgical fiducial marker system 10 can generally include a spinous process clamp 100 and a surgical fiducial marker positioner 200. Spinous process clamp 100 can be clamped to one of various spinous processes 2 along the spine 1 of a patient. Surgical fiducial marker positioner 200 can be coupled to spinous process clamp 100 and can be configured to position multiple surgical fiducial markers 210 with respect to the patent spine 1.

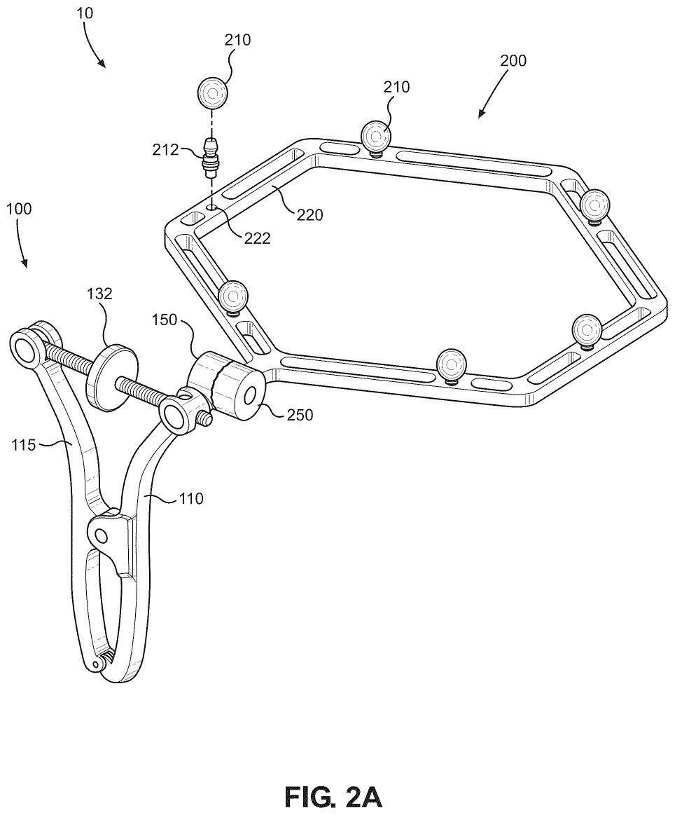

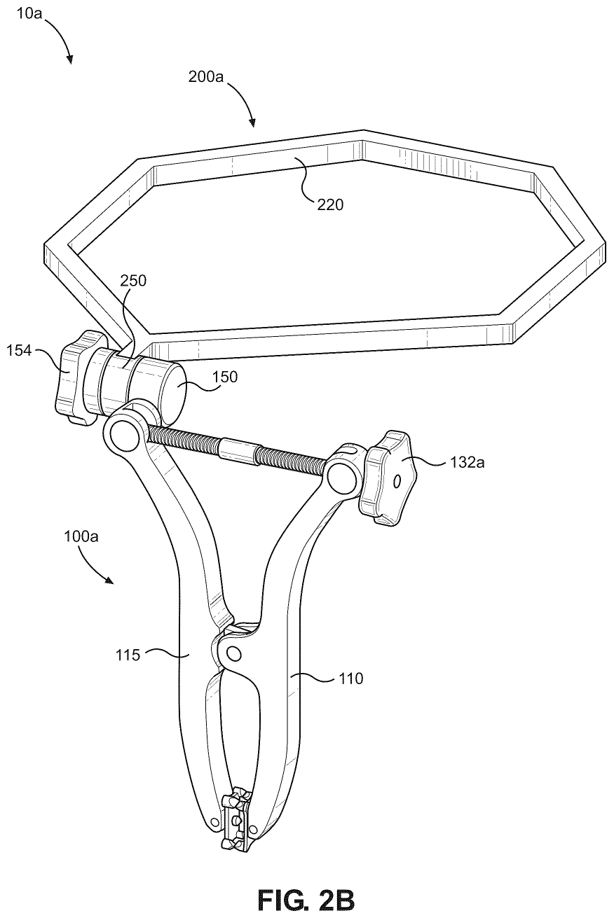

Continuing with FIG. 2A, an example surgical fiducial marker system having a spinous process clamp is shown in front perspective view. Again, surgical fiducial marker system 10 can generally include a spinous process clamp 100 and a surgical fiducial marker positioner 200. In various embodiments, spinous process clamp 100 can include a first arm 110, a second arm 115, a thumbwheel 132, and a clamp coupling component 150, among various other components and features set forth in greater detail below. While clamp coupling component 150 is shown as being located proximate the top of first arm 110, it will be readily appreciated that this coupling component can alternatively be located at various other places on spinous process clamp 100, such as proximate the top of second arm 115 for example.

Surgical fiducial marker positioner 200 can be configured to position multiple surgical fiducial markers 210 with respect to a patient and can be coupled to spinous process clamp 100 by way of a positioner coupling component 250 that can be configured to interact with clamp coupling component 150 of the clamp, as detailed below. In various embodiments, surgical fiducial markers 210 can include infrared reflective balls or other retroreflective spheres, infrared-emitting diodes, or other items suitable for use with surgical optical tracking systems. Each surgical fiducial marker 210 can be coupled to an arm segment 220 of surgical fiducial marker positioner 200 using a threaded coupler 212 that can be inserted into an opening of the fiducial marker as well as an opening 222 along the arm segment 220.

As shown in FIG. 2A, surgical fiducial marker positioner 200 can include multiple arm segments 220 arranged into an irregular polygon shape, such as a hexagon. Each arm segment 220 can include an opening 222 for a surgical fiducial marker 210, which can be removably coupled to the opening 222. The locations of openings 222 can form an asymmetrical pattern such that installation of surgical fiducial markers 210 into all of the openings then results in an asymmetrical pattern that will not cause ambiguity or confusion with the optical tracking system reading the locations of the fiducial markers. Although six surgical fiducial markers and six arm segments 220 arranged into a hexagon shape are shown for purposes of illustration, it will be understood that a suitable surgical fiducial marker positioner may also have more or fewer fiducial markers arranged along more or fewer arm segments arranged into other shapes. Such other shapes can include rectangles, pentagons, octagons, for example, among other possible shapes. Further details regarding surgical fiducial marker positioner 200 can be found in related U.S. patent application Ser. No. ______/______,______ entitled “SURGICAL FIDUCIAL MARKER POSITIONER,” which application is again hereby incorporated by reference in its entirety.

FIG. 2B illustrates in front perspective view an example alternative surgical fiducial marker system with a spinous process clamp. Alternative surgical fiducial marker system 10a can be substantially similar to surgical fiducial marker system 10 in FIG. 2A, only with some variations to different components. Alternative fiducial marker system 10a can include an alternative spinous process clamp 100a and alternative surgical fiducial marker positioner 200a that both have minor variations from spinous process clamp 100 and surgical fiducial marker positioner 200. For example, alternative spinous process clamp 100a can include thumbwheel 132a located outside of first arm 110 and second arm 115 rather than between these arms, and this alternative thumbwheel 132a can have multiple nodules forming a star-shape rather than the circular shape of thumbwheel 132 above. Also, clamp coupling component 150 can be located proximate the top of second arm 115, and a thumbwheel and pin arrangement 154 can be used to tighten positioner coupling component 250 against clamp coupling component 150, as noted below. As another example, alternative surgical fiducial marker positioner 200a can have seven arm segments 220 rather than the six arm segments 220 shown for surgical fiducial marker positioner 200 in FIG. 2A. Other minor variations in system features are also possible.

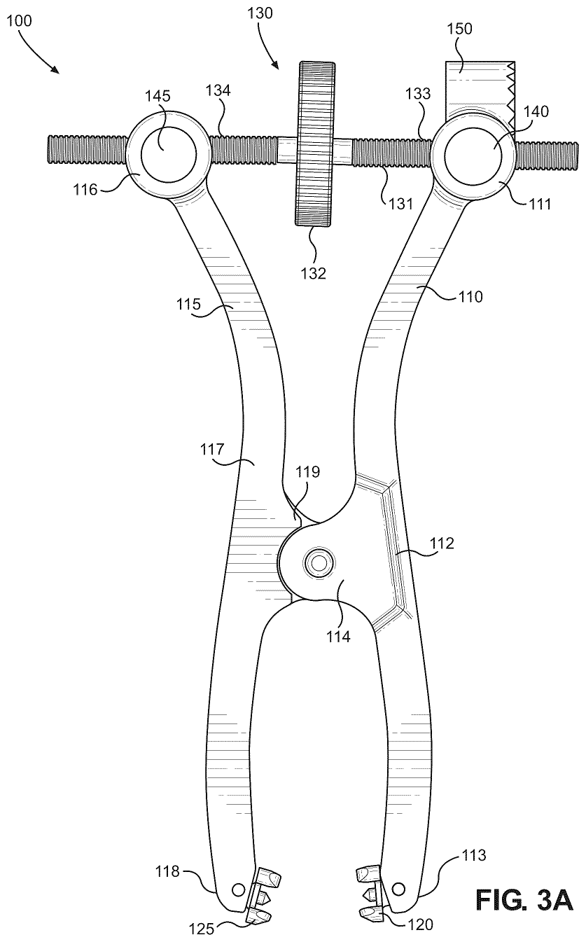

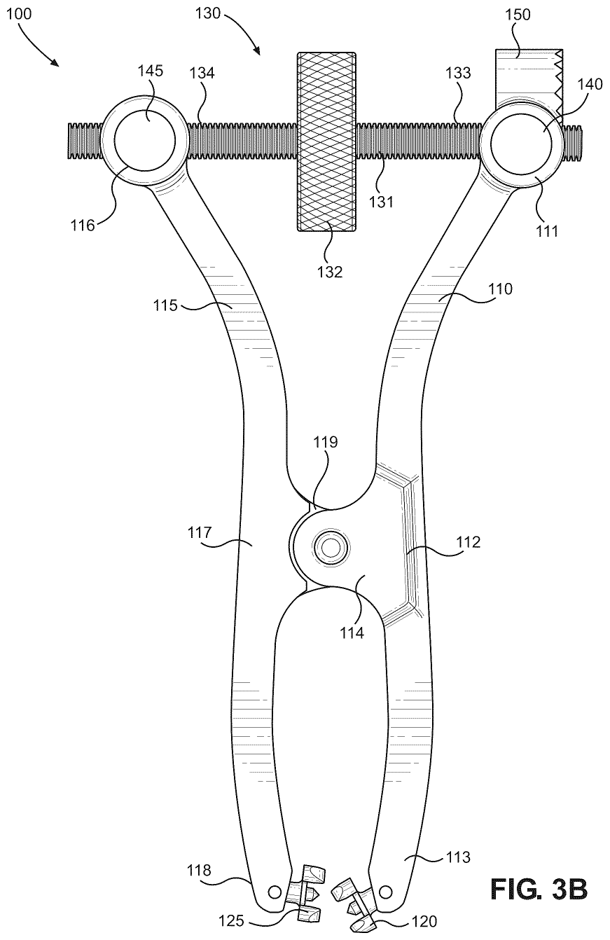

Focusing now on FIGS. 3A and 3B, an example spinous process clamp is shown in front elevation views in open and closed positions respectively. Spinous process clamp 100 can include a first arm 110, a second arm 115, a first gripping component 120, a second gripping component 125, an adjustable spacing arrangement 130 including a first receiving bearing 140 and a second receiving bearing 145, and a clamp coupling component 150, among other possible components and features. First arm 110 can have a top distal end 111, a midsection 112, a bottom distal end 113, and a first pivoting feature 114, while second arm 115 can similarly have a top distal end 116, a midsection 117, a bottom distal end 118, and a second pivoting feature 119. Midsection 117 of second arm 115 can be pivotally coupled to midsection 112 of first arm 110 by way of a pivoting arrangement between first and second pivoting features 114, 119. Clamp coupling component 150 can be coupled to first arm 110 proximate its top distal end 111 and can be configured to couple spinous process clamp 100 to a separate surgical component, such as a surgical fiducial marker positioner 200, as noted above.

First gripping component 120 can be located proximate bottom distal end 113 of first arm 110 and can be configured to grip a side of a spinous process of a patient, while second gripping component 125 can similarly be located proximate bottom distal end 118 of second arm 115 and can be configured to grip a side of the spinous process opposite the side gripped by the first gripping component. Pivoting first and second arms 110, 115 with respect to each other can adjust a distance between first and second gripping components 120, 125. As shown in FIG. 3A, first and second arms 110, 115 of spinous process clamp 100 are pivoted to put the clamp into an “open” position such that first and second gripping components 120, 125 are spaced relatively far apart. Conversely, FIG. 3B depicts first and second arms 110, 115 of spinous process clamp 100 as pivoted to put the clamp into a “closed” position such that first and second gripping components 120, 125 are spaced relatively close together. While specific open and closed positions are shown in FIGS. 3A and 3B, it will be readily appreciated that spinous process clamp 100 can be adjusted to a wide variety of positions having different exact distances between first and second gripping components 120, 125.

The exact spacing or distance between first and second gripping components 120, 125 can be facilitated by adjustable spacing arrangement 130, which can be coupled to first arm 110 proximate top distal end 111 and to second arm 115 proximate top distal end 116. Adjustable spacing arrangement 130 can include threaded bar 131, thumbwheel 132 located proximate the center of the threaded bar, and first and second receiving bearings 140, 145 located on opposite sides of the thumbwheel toward the distal ends of the threaded bar. In some arrangements thumbwheel 132 can be integrally formed with threaded bar 131, while in other arrangements this thumbwheel can be firmly attached to the threaded bar as a separate component. Each of first and second receiving bearings 140 and 145 can be located at and rotatable within top distal ends 111 and 116 respectively. Each of first and second receiving bearings 140 and 145 can also have an internal threaded portion configured to receive respective first and second threaded portions 133, 134 of threaded bar 131 and pass those respective threaded portions therethrough when the threaded bar is rotated, such as by thumbwheel 132.

Adjustable spacing arrangement 130 can be configured to maintain a pivoted position of first and second arms 110, 115 with respect to each other. For example, external threads along first and second threaded portions 133, 134 of threaded bar 131 can engage with internal threads of respective first and second receiving bearings 140, 145 such that these receiving bearings do not slide along the threaded bar, while a tight fit between these receiving bearings and their respective top distal ends 111, 116 of first and second arms 110, 115 prevent relative pivoting or rotational movement of the arms. Rotational adjustment of adjustable spacing arrangement 130 can result in pivoting first and second arms 110, 115 with respect to each other. For example, rotation of thumbwheel 132 in one direction can result in rotation of first and second threaded portions 133, 134 of threaded bar 131, which can in turn result in movement of first and second receiving bearings 140, 145 along their respective threaded portions, which can then result in movement of top distal ends 111, 116 of first and second arms 110, 115.

Thumbwheel 132 can be operated manually, such as by a surgeon, for example, in some embodiments, or can alternatively be automatically controlled by one or more robotic components in some situations. In some arrangements, rotation of thumbwheel 132 in a forward or “clamp opening” direction can result in moving receiving bearings 140, 145 and top distal ends 111, 116 toward the thumbwheel, which can then result in increasing the distance between first and second gripping components 120, 125 due to the pivoting arrangement of first and second arms 110, 115. In such arrangements, rotation of thumbwheel 132 in a reverse or “clamp closing” direction can then result in moving receiving bearings 140, 145 and top distal ends 111, 116 away from the thumbwheel, which can then result in decreasing the distance between first and second gripping components 120, 125 due to arm pivoting.

As noted above, thumbwheel 132 can take alternative forms and can be located at other places along threaded bar 131. FIG. 2B depicts a star-shaped thumbwheel 132a having five nodules, for example, and this thumbwheel 132a can be located on threaded bar 131 on or at its distal end outside of first arm 110. More or fewer than five nodules of a star-shape are also possible, other alternative thumbwheel shapes are also possible, and the location of the thumbwheel can be on or at the other end of threaded bar 131 or at any other suitable location along the threaded bar. Other mechanisms or features for readily adjusting the spacing between first and second arms 110, 115 are also possible, as will be readily appreciated.

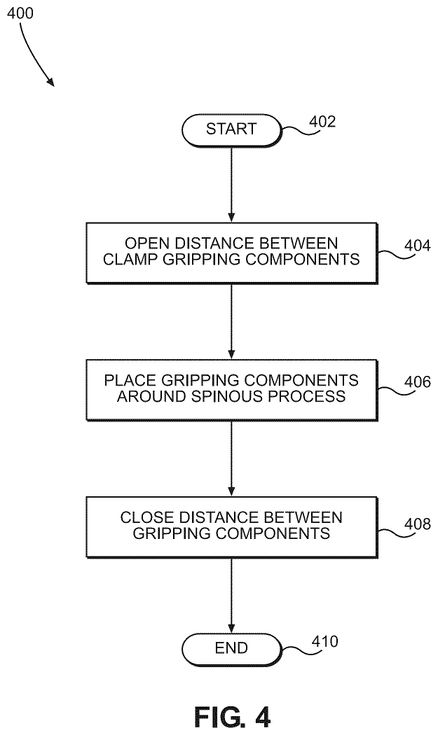

Moving next to FIG. 4, a flowchart of an example summary method of attaching a spinous process clamp to a spinous process is provided. Summary method 400 can represent one broad aspect of overall methods of use for a spinous process clamp, and it will be understood that various other steps, features, and details of such a broad aspect and overall methods of use are not provided here for purposes of simplicity. After a start step 402, a first process step 404 can involve opening or increasing a distance between gripping components of a spinous process clamp. This can involve rotating the thumbwheel of the clamp in a forward or clamp opening direction, for example. Step 404 can be manually or automatically performed, such as where a separate robotic system can be configured to operate a thumbwheel of the spinous process clamp.

At the following process step 406, the gripping components of the spinous process clamp can be placed around a spinous process of a patient. This can involve placing the gripping components and the overall spinous process clamp in such a way relative to the spinous process so as to facilitate a firm gripping or clamping onto the spinous process. Step 406 can be manually or automatically performed, such as where a separate robotic system can be configured to move and place the spinous process clamp.

The next process step 408 can involve closing or decreasing the distance between the gripping components. This can involve rotating the thumbwheel of the clamp in a reverse or clamp closing direction, for example. The distance between gripping components can be closed or decreased until they firmly grip the spinous process and affix the spinous process clamp in place relative to the patient. Step 408 can be manually or automatically performed, such as where a separate robotic system can be configured to operate a thumbwheel of the spinous process clamp. The method can then end at end step 410.

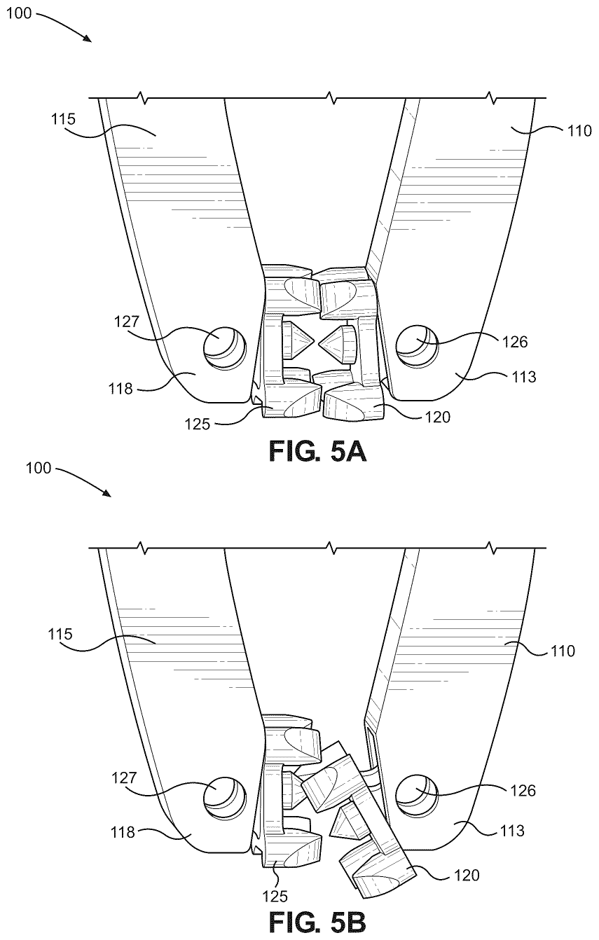

Transitioning now to FIGS. 5A and 5B, an example pair of gripping components coupled to spinous process clamp arms are shown in front elevation views in one position and an alternative position respectively. First gripping component 120 can be coupled to first arm 110 at its bottom distal end 113, while second gripping component 125 can similarly be coupled to second arm 115 at its bottom distal end 118. In various arrangements, first and second gripping components 120, 125 can be identical or substantially similar. As shown, both first and second gripping components 120, 125 can be arranged such that they face each other so as to facilitate clamping onto and gripping of a spinous process that may be placed between them.

In some embodiments, one or both of first and second gripping components 120, 125 can be arranged to move relative its respective arm 110, 115, such as to facilitate better gripping and clamping on a given spinous process. For example, first gripping component 120 can be pivotally coupled to first arm 110 about an axis through first opening 126 in the first arm, while second gripping component 125 can be pivotally coupled to second arm 115 about an axis through second opening 127 in the second arm. This can be accomplished by way of pivot pins (not shown) inserted through first and second openings 126, 127 and portions of respective gripping components 120, 125, which pivot pins can function to hold the gripping components in place against respective first and second arms 110, 115 while also allowing the gripping components to pivot with respect to the arms. As shown, each of first and second gripping components 120, 125 can rotate about 10-15 degrees with respect to first and second arms 110, 115 respectively. Other ranges of motion are also possible, and it is also contemplated that one or both of first and second gripping components 120, 125 can have multiple axes of rotation with respect to first and second arms 110, 115 to facilitate further relative movement.

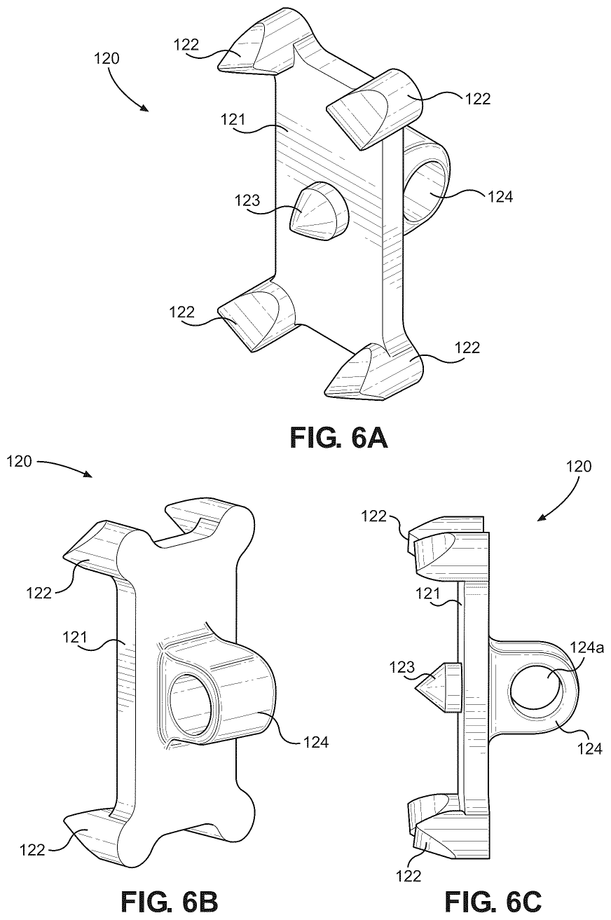

FIGS. 6A-6C illustrate an example gripping component for a spinous process clamp in front perspective, rear perspective, and side elevation views respectively. Again, gripping component 120 can be substantially similar or identical to gripping component 125 in the foregoing examples. Gripping component 120 can include a main body 121 defining an outer perimeter having a plurality of outer gripping teeth 122 distributed thereabout, as well as a central region having one or more central gripping teeth 123 extending therefrom. As shown, four outer gripping teeth 122 can extend from each corner of rectangular main body 121, while one central gripping tooth 123 can extend from the center of the main body. In various embodiments, gripping teeth 122, 123 can be about 4-5 mm wide and about 5-15 mm tall, although other dimensions are also possible.

As shown, gripping teeth 122, 123 can all extend in the same direction from main body 121 of gripping component 120, while pivot extension 124 can extend in the opposite direction from the main body. Pivot extension 124 can have an opening 124a therethrough to accommodate a pin inserted therethrough when the pivot extension is inserted into a slot at the bottom distal end of an arm of a spinous process clamp. For example, pivot extension 124 can be inserted into a slot at the interior of bottom distal end 113 of first arm 110 in the above example. A pivot pin (not shown) can then be inserted through both opening 124a in pivot extension 124 and opening 126 of first arm 110 to hold gripping component 120 in place against the first arm and also allow the gripping component to pivot with respect to the first arm. In some arrangement gripping component 120 can be integrally formed from machined surgical steel, although other compositions and materials may also be used.

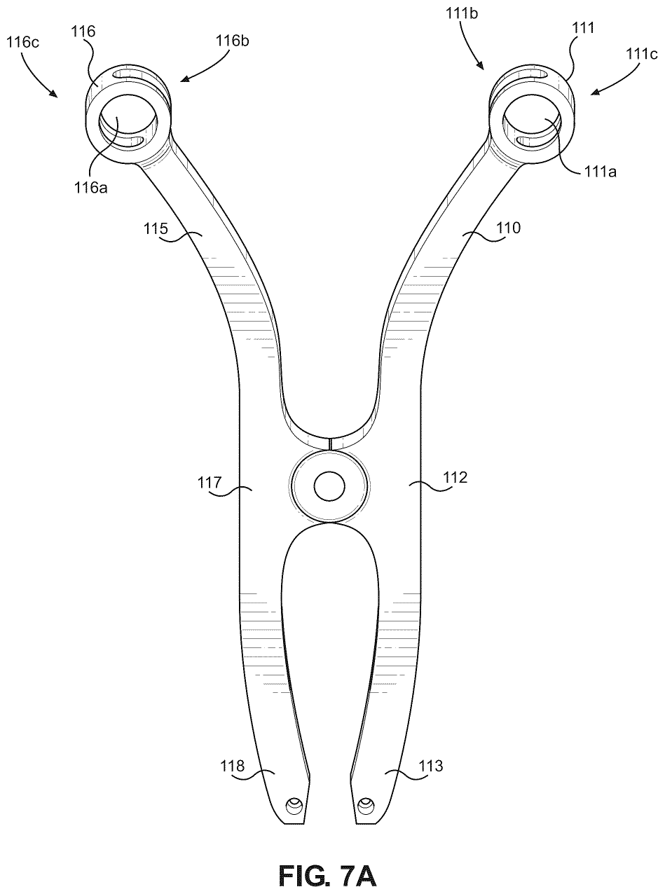

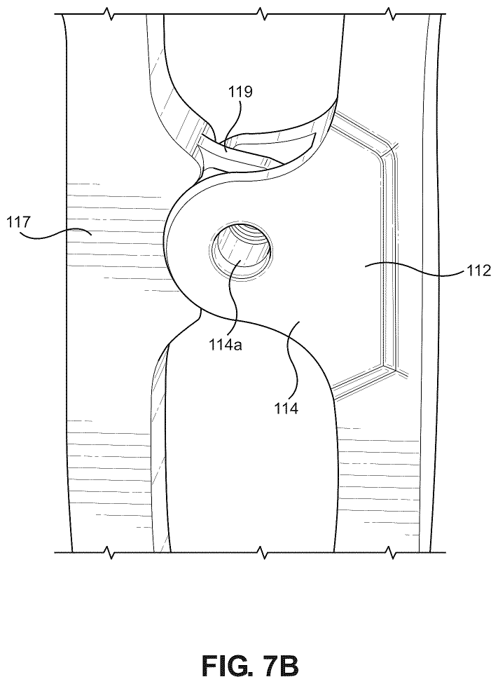

FIG. 7A illustrates in front elevation view an example pair of arms for a spinous process clamp, while FIG. 7B illustrates in front elevation view an example coupling arrangement for this pair of arms. Again, first arm 110 can have a top distal end 111, a midsection 112, a bottom distal end 113, and a first pivoting feature 114, while second arm 115 can similarly have a top distal end 116, a midsection 117, a bottom distal end 118, and a second pivoting feature 119. Top distal ends 111, 116 of first and second arms 110, 115 can include hollow regions 111a, 116a configured to hold rotational bearings therein, as noted above. In addition, top distal ends 111, 116 can also include inside slots 111b, 116b at interior sidewalls thereof, as well as outside slots 111c, 116c at exterior sidewalls thereof. Inside slots 111b, 116b and outside slots 111c, 116c can be arranged to accommodate first and second threaded portions of a threaded bar passing therethrough respectively as the top distal ends 111, 116 move toward or away from each other along the threaded bar when first and second arms 110, 115 pivot.

As noted above, midsection 117 of second arm 115 can be pivotally coupled to midsection 112 of first arm 110 by way of a pivoting arrangement between a first pivoting feature 114 of the first arm and a second pivoting features 119 of the second arm. In some arrangements, second pivoting feature 119 can extend inward from second arm 115 and fit between multiple portions of first pivoting feature 114 that extend inward from first arm 110. Each of these portions of first pivoting feature 114 can have an opening 114a therethrough to facilitate the insertion of a pivot pin (not shown), and second pivoting feature 119 can also have an opening (not shown) therethrough to facilitate insertion of the pivot pin. Use of such a pivot pin can function to hold first and second arms 110, 115 in place against each other while also allowing the arms to pivot with respect to each other about the pin.

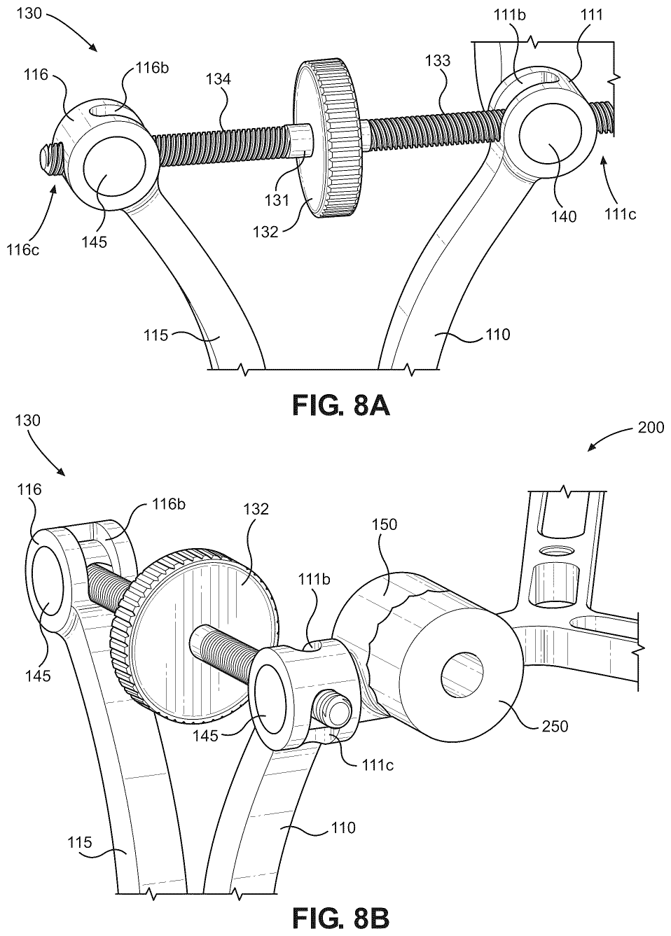

Referring next to FIGS. 8A and 8B, an example adjustable spacing arrangement for a spinous process clamp is shown in front perspective and side perspective views respectively. As noted above, adjustable spacing arrangement 130 can include threaded bar 131, thumbwheel 132 located proximate the center of the threaded bar, first and second threaded portions 133, 134 of the threaded bar, and first and second receiving bearings 140, 145 located on opposite sides of the thumbwheel toward the distal ends of the threaded bar. First and second receiving bearings 140, 145 can be located at and rotatable within top distal ends 111, 116 of first and second arms 110, 115 respectively. Inside slots 111b, 116b and outside slots 111c, 116c in top distal ends 111, 116 of first and second arms 110, 115 can be arranged to accommodate first and second threaded portions 133, 134 of threaded bar 131 passing therethrough respectively as the top distal ends move toward or away from each other along the threaded bar.

In some arrangements, threaded bar 131 can be a two-way custom screw with right-hand and left-hand threads on either side of thumbwheel 132. For example, first threaded portion 133 can have a right-hand thread while second threaded portion 134 can have a left-hand thread. Accordingly, a threaded internal opening of first receiving bearing 140 can be configured to receive the right-hand thread of first threaded portion 133, while a threaded internal opening of second receiving bearing 145 can be configured to receive the left-hand thread of second threaded portion 134. In various embodiments, the pitch of each thread can be about 1.5 mm, although other pitches can also be used.

Rotation of thumbwheel 132 in a forward direction can result in rotating threaded bar 131 and its first and second threaded portions 133, 134 in a forward direction, which in turn moves the first and second receiving bearings 140, 145 inward along the threaded bar toward the thumbwheel. Similarly, rotation of thumbwheel 132 in a reverse direction can result in rotating threaded bar 131 and its first and second threaded portions 133, 134 in a reverse direction, which in turn moves the first and second receiving bearings 140, 145 outward along the threaded bar away from the thumbwheel. In either directional motion the receiving bearing 140, 145 can both move equally, such that top distal ends 111, 116 of first and second arms 110, 115 both move equally as well.

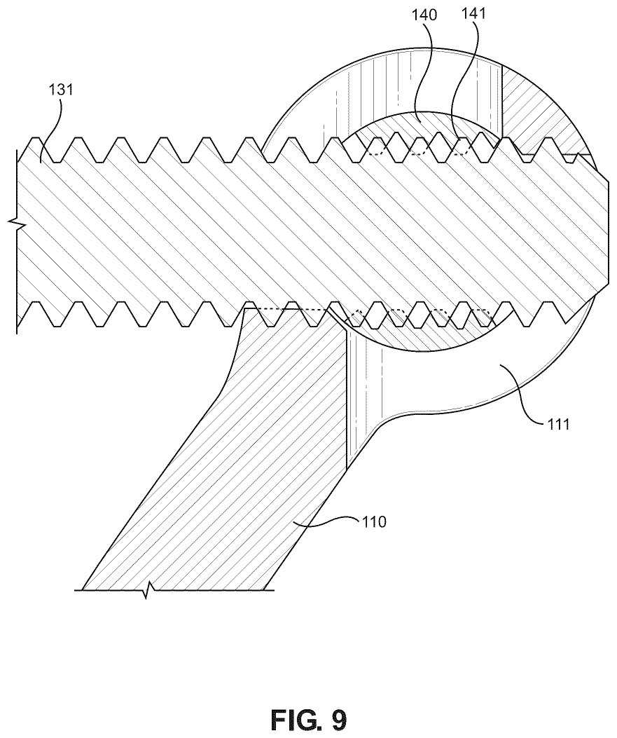

Continuing with FIG. 9 an example receiving bearing within a spinous process clamp is shown in front cross-section view. Receiving bearing 140 can be identical or substantially similar to receiving bearing 145 noted above and can be cylindrically shaped and sized to fit snugly within a suitably sized cylindrically shaped hollow region within top distal end 111 of an arm 110 of the spinous process clamp. Receiving bearing 140 can then rotate within this hollow region within top distal end 111, since the relative orientation of the top distal end will rotate with respect to the receiving bearing as the top distal end and receiving bearing move together back and forth along threaded bar 131. To facilitate such movement, receiving bearing 140 can have a central opening extending longitudinally therethrough, and this central opening can have an internal threaded portion 141 configured to receive and mate with the threaded bar.

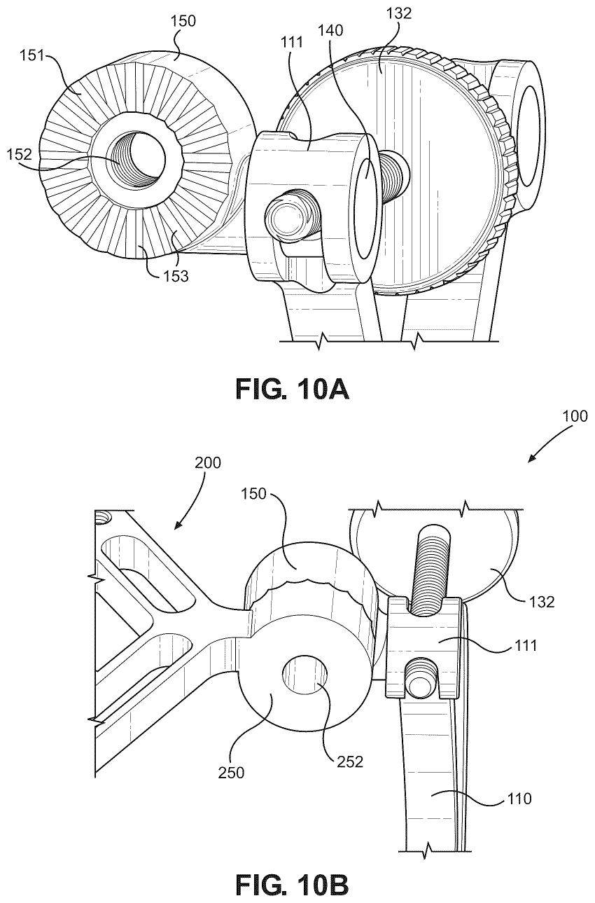

FIG. 10A illustrates in side perspective view an example clamp coupling component for a spinous process clamp, while FIG. 10B illustrates in top perspective view the clamp coupling component of FIG. 10A rotationally coupled to a positioner coupling component for a surgical fiducial marker positioner. As noted above, clamp coupling component 150 can be attached or otherwise coupled to top distal end 111 of first arm 110, among other possible locations at spinous process clamp 100. Clamp coupling component 150 can define a cylindrical shape having a vertically oriented circular mating face 151 and a threaded central opening 152 extending at least partially therethrough. Mating face 151 can have a plurality of ridges 153 that can be arranged in an axial pattern for mating with a similar axial pattern of ridges on a mating face of positioner coupling component 250. In addition to having a corresponding mating face that is configured to mate and operate with mating face 151, positioner coupling component 250 can similarly have a threaded central opening 252 arranged to align with the threaded central opening 152 of clamp coupling component 150 when both mating faces are engaged.

In various arrangements, clamp coupling component 150 can be affixed to and can be stationary with spinous process clamp 100, while positioner coupling component 250 can be configured to rotate with respect to the clamp coupling component. Rotation can be about a horizontal axis that extends through the centers of threaded central openings 152 and 252, such that the position of entire surgical fiducial marker positioner 200 is rotated about this axis. As will be readily appreciated, rotation of positioner coupling component 250 with respect to a stationary clamp coupling component 150 can result in the relative rotation of its mating face with mating face 151 of the clamp coupling component.

Ridges 153 on mating face 151 can fit snugly between corresponding ridges on the mating face of rotating positioner coupling component 250, such that multiple discrete rotational orientations can be achieved. For example, where clamp coupling component 150 has a mating face 151 with ten axially arranged ridges 153, then positioner coupling component 250 can have a matching mating face with ten similarly axially arranged ridges, and this can facilitate at least ten different discrete rotational positions of surgical fiducial marker positioner 200 with respect to the spinous process clamp 100. Of course, more or fewer axially arranged ridges are also possible, and clamp coupling component 150 as shown can have twenty such axially arranged ridges 153 for twenty different discrete rotational positions in this particular illustrative example.

In some embodiments, a thumbscrew, wing nut, or other coupling arrangement (not shown) can be used within threaded internal openings 151, 251 to loosen and tighten positioner coupling component 250 with respect to clamp coupling component 150. One possible such coupling arrangement can involve the thumbwheel and pin arrangement 154 shown in FIG. 2B above, which can involve a threaded pin inserted into threaded central openings 252 and 152 to tighten positioner coupling component 250 against clamp coupling component 150. As will be readily appreciated, loosening such a coupling arrangement can allow for the ready rotational adjustment of surgical fiducial marker positioner 200 with respect to spinous process clamp 100, while tightening such a coupling arrangement can result in affixing or setting a discrete rotational position of the surgical fiducial marker positioner with respect to the spinous process clamp.

While clamp coupling component 150 and positioner coupling component 250 are shown as being vertically oriented with respect to spinous process clamp 100 in FIGS. 10A-B and other illustrative examples herein, it will be readily appreciated that these coupling components can be oriented horizontally or otherwise, such that relative rotation can be about a differently oriented axis. It is also contemplated that more than one rotational axis can be used for adjusting the positioning of surgical fiducial marker positioner 200 in some embodiments. For example, a secondary mating coupling component arrangement can allow for rotation of the surgical fiducial marker positioner 200 with respect to the spinous process clamp 100 about a vertical axis as well as a horizontal axis.

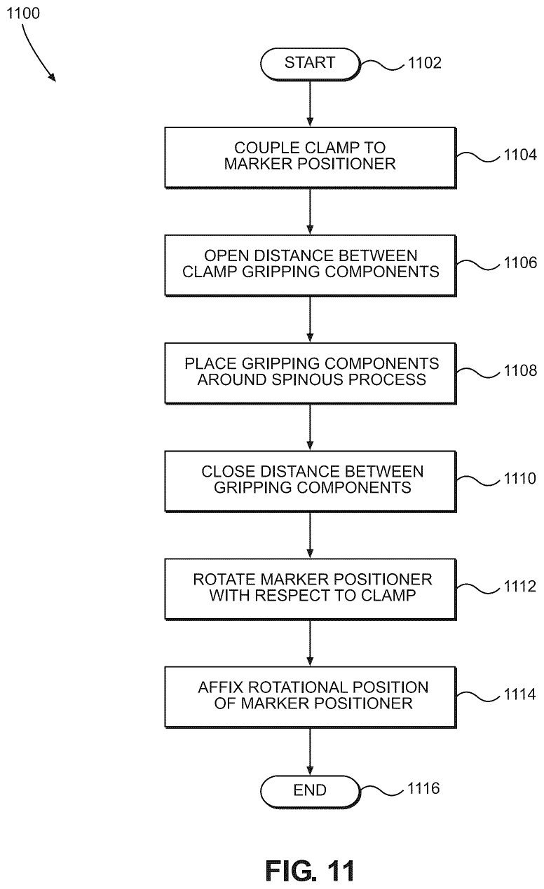

Next, FIG. 11 provides a flowchart of an example detailed method 1100 of using a spinous process clamp. Detailed method 1100 can represent one possible way of using a spinous process clamp, and it will be understood that various other steps, features, and details of such a detailed method are not provided here for purposes of simplicity. After a start step 1102, a first process step 1104 can involve coupling a spinous process clamp to a surgical fiducial marker positioner. This can be a rotational coupling, such as that which is set forth above. Step 1102 can be manually or automatically performed, such as where a separate robotic system can be configured to couple a spinous process clamp to a surgical fiducial marker positioner.

At a following process step 1106, a distance between gripping components of a spinous process clamp can be opened or increased. This can involve rotating a thumbwheel of the clamp in a forward or clamp opening direction, for example. Step 1106 can be manually or automatically performed, such as where a separate robotic system can be configured to operate a thumbwheel of the spinous process clamp.

At subsequent process step 1108, the gripping components of the spinous process clamp can be placed around a spinous process of a patient. This can involve placing the gripping components and the overall spinous process clamp in such a way relative to the spinous process so as to facilitate a firm gripping or clamping onto the spinous process. Step 1108 can be manually or automatically performed, such as where a separate robotic system can be configured to move and place the spinous process clamp.

The next process step 1110 can involve closing or decreasing the distance between the gripping components. This can involve rotating the thumbwheel of the clamp in a reverse or clamp closing direction, for example. The distance between gripping components can be closed or decreased until they firmly grip the spinous process and affix the spinous process clamp in place relative to the patient. Step 1110 can be manually or automatically performed, such as where a separate robotic system can be configured to operate a thumbwheel of the spinous process clamp.

At a following process step 1112, the surgical fiducial marker positioner can be rotated with respect to the spinous process clamp. This can be done while the spinous process clamp is coupled to the surgical fiducial marker positioner and the spinous process clamp is affixed in place relative to the patient. Also, this can involve loosening any couplings or other attachments between these items and then rotating or swiveling the surgical fiducial marker positioner about an axis through the couplings, for example. Such couplings can be a clamp coupling component and a positioner coupling component as detailed above. Step 1112 can be manually or automatically performed, such as where a separate robotic system can be configured to rotate the surgical fiducial marker positioner relative to the spinous process clamp.

Subsequent process step 1114 can involve affixing the rotational position of the surgical fiducial marker positioner relative to the spinous process clamp. This can involve the use of thumbscrew or wing nut, for example, which can be tightened on a clamp coupling component as noted above. Step 1114 can be manually or automatically performed, such as where a separate robotic system can be configured to affix the surgical fiducial marker positioner in place to the spinous process clamp. The method can then end at end step 1116.

For foregoing method 1100, it will be appreciated that not all process steps are necessary, and that other process steps may be added in some arrangements. For example, changing the clamp from one spinous process to another might take place in some arrangements. Furthermore, the order of steps may be altered in some cases, and some steps may be performed simultaneously. For example, step 1104 may be performed later in the process in some cases. Although known process steps are provided for the various techniques in method 1100, it will be appreciated that any other suitable similar method for using a spinous process clamp can also be used. Other variations and extrapolations of the disclosed methods will also be readily appreciated by those of skill in the art.

### Surgical Spinous Process Clamps

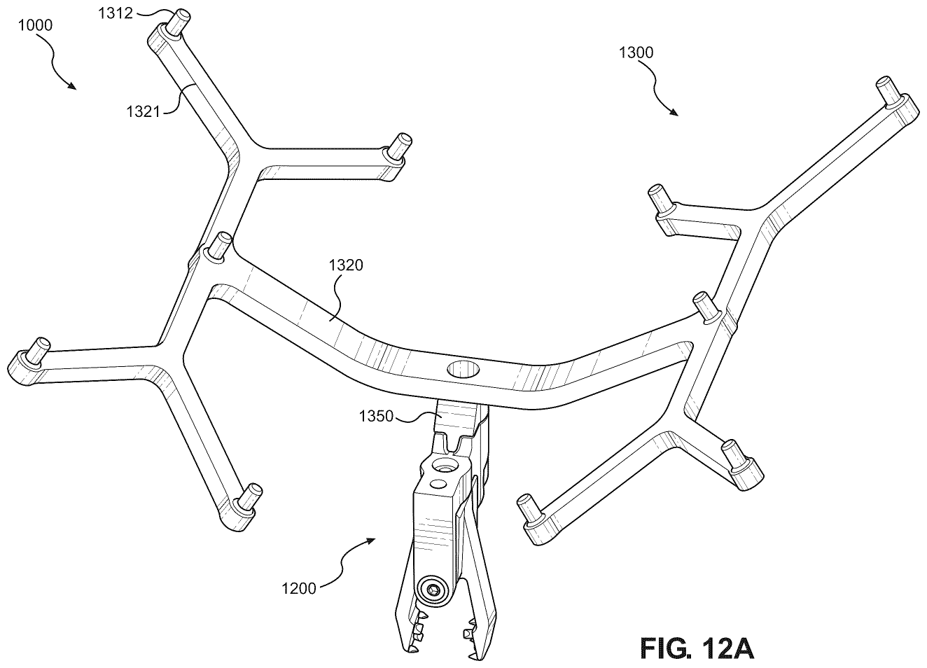

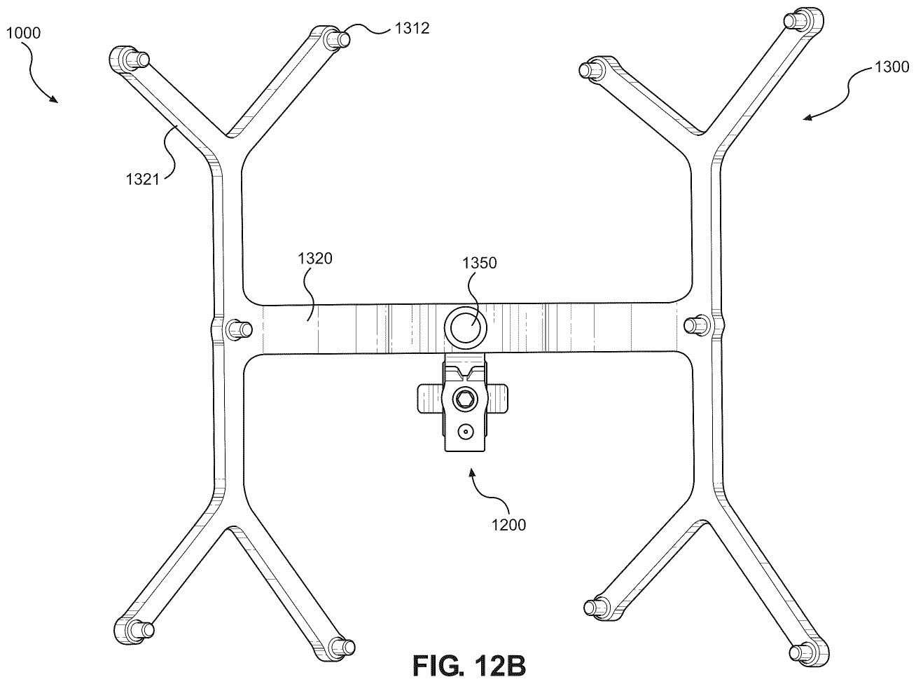

Transitioning now to FIGS. 12A and 12B, an example alternative surgical fiducial marker system having a surgical spinous process clamp and a magnetically couplable fiducial marker array is illustrated in front perspective and top plan views respectively. Alternative surgical fiducial marker system 1000 can generally include a surgical spinous process clamp 1200 and a magnetically couplable fiducial marker array 1300. It will be understood that surgical spinous process clamp 1200 can be different in various ways from spinous process clamp 100 above while still performing some of the same functions. Similar to spinous process clamp 100 above (which can also be considered surgical in nature), for example, surgical spinous process clamp 1200 can be used to clamp to one of various spinous processes along the spine of a patient, such as during a surgery.

It will also be understood that magnetically couplable fiducial marker array 1300 can be different in various ways from surgical fiducial marker positioner 200 above while still performing some of the same functions. Similar to surgical fiducial marker positioner 200 above (which can also be considered a fiducial marker array), magnetically couplable fiducial marker array 1300 can be used to position surgical fiducial markers in space while coupled to surgical spinous process clamp 1200. These surgical fiducial markers (not shown) can be identical or substantially similar to surgical fiducial markers 210 above and can be coupled to magnetically couplable fiducial marker array 1300 by way of fiducial marker couplers 1312 located at each position where a surgical fiducial marker should be coupled.

Magnetically couplable fiducial marker array 1300 can include a rigid body 1320 configured to support a plurality of surgical fiducial markers (not shown) at fixed positions relative to each other along the rigid body by way of fiducial marker couplers 1312 to form a fixed positional arrangement of surgical fiducial markers that can be asymmetrical, as shown. Magnetically couplable fiducial marker array 1300 can be a full array having surgical fiducial markers at rigid body branches 1321 on both sides of magnetic coupling arrangement 1350 that couples the full array to surgical spinous process clamp 1200. Each side or half of magnetically couplable fiducial marker array 1300 can include a support portion of the rigid body 1320 that can be arranged along a surgical fiducial marker support plane, such that all surgical fiducial markers on each side are aligned along the same plane. The fixed positional arrangement of all surgical fiducial markers (located at fiducial marker couplers 1312 when installed) can be asymmetrical. In some arrangements, each half or side of magnetically couplable fiducial marker array 1300 can also include its own fixed positional arrangement of its half or side of the surgical fiducial markers that can also by asymmetrical.

As shown, magnetically couplable fiducial marker array 1300 can include coupling arrangement 1350 integrally formed with, affixed to, or otherwise coupled to rigid body 1320. Coupling arrangement 1350 configured to couple the fiducial marker array 1300 to a separate surgical device (e.g., surgical spinous process clamp 1200) at a first fixed position relative to the separate surgical device. Other aspects of magnetically couplable fiducial marker array 1300 are set forth in greater detail in related U.S. Provisional patent application Ser. No. ______/______,______, titled “MAGNETICALLY COUPLABLE FIDUCIAL MARKER ARRAYS,” which application is again hereby incorporated by reference in its entirety herein.

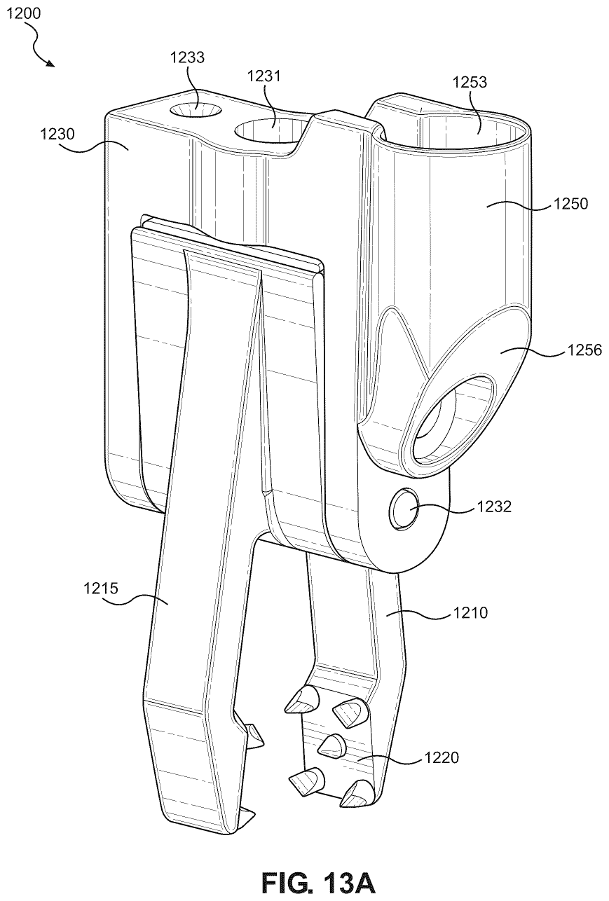

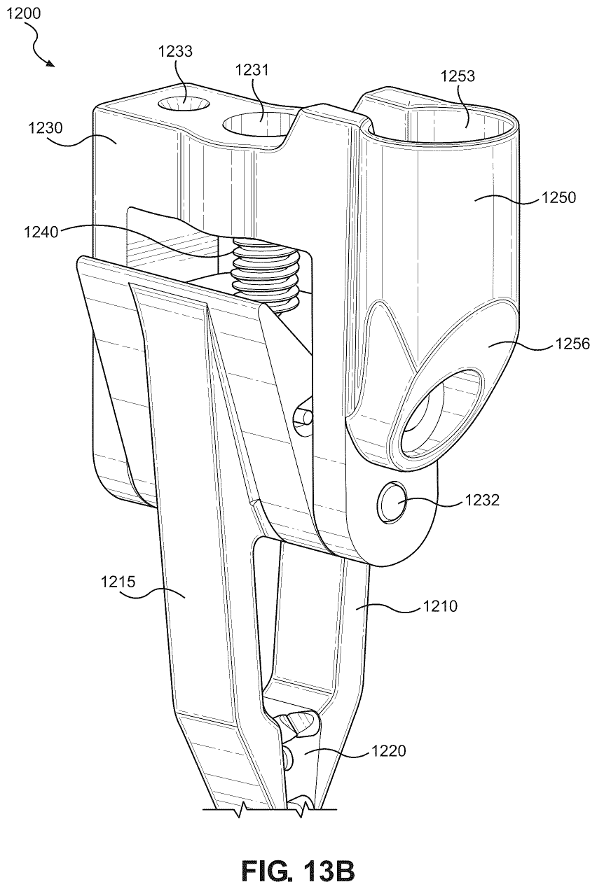

Moving next to FIGS. 13A and 13B, an example surgical spinous process clamp is shown in front perspective views in an open position and a closed position respectively. In some arrangements, surgical spinous process clamp 1200 can be a smaller clamp than spinous process clamp 100 above. While surgical spinous process clamp 1200 can function in a similar manner to clamp 100 in that it can clamp onto a spinous process of a patient and can also have a coupling arrangement to facilitate coupling to a fiducial marker array, this surgical spinous process clamp can have several different components and features.

Surgical spinous process clamp 1200 can have a first arm 1210 and second arm 1215, which can have first gripping and second gripping components 1220 located proximate the bottom portions of the first and second arms respectively and configured to grip first and second sides of a spinous process of a patient. Each arm 1210, 1215 can have a top portion, a bottom portion, and a midsection configured to rotate about pin 1232 in first and second rotational directions respectively. Surgical spinous process clamp 1200 can also have a main body 1230 having a top region, a first side region, a second side region opposite the first side region, and a pin 1232 extending between the first and second side regions. Main body 1230 can also have a reference divot 1233 at a surface thereof to help facilitate calibration of associated surgical fiducial markers, such as those that may be coupled to a surgical probe having its tip inserted into the reference divot.

Shaft 1240 can extend through the top region of main body 1230, and this shaft can be threaded to facilitate forcing movement up and down of an internal traveling component, as shown in greater detail below. Clamp coupling arrangement 1250 can be coupled to the main body 1250, and this clamp coupling arrangement can be configured to couple the spinous process clamp 1200 to a separate surgical device, such as a fiducial marker array. Clamp coupling arrangement 1250 can have one or more internal magnetic components to facilitate a magnetic coupling with the separate surgical device. Opening 1253 atop clamp coupling arrangement 1250 can be sized and shaped to receive a mating component from the separate surgical device, which can be a fiducial marker array having corresponding mating magnetic components. Cover 1256 can be coupled to a side of main body 1230 and can be arranged to help contain the internal magnetic components within an internal cavity within clamp coupling arrangement 1250.

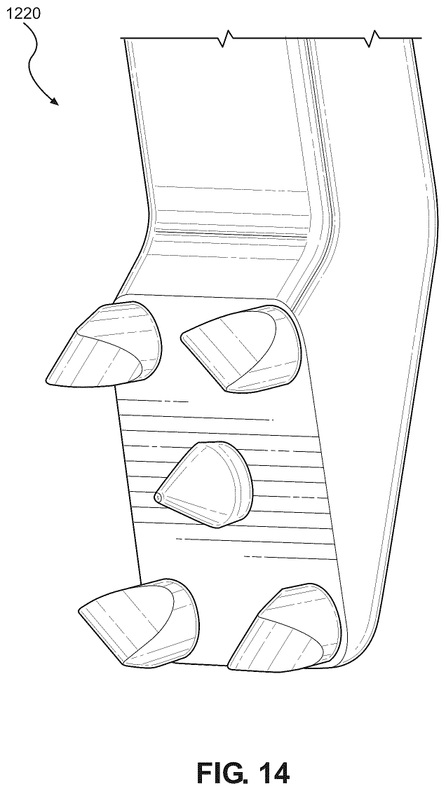

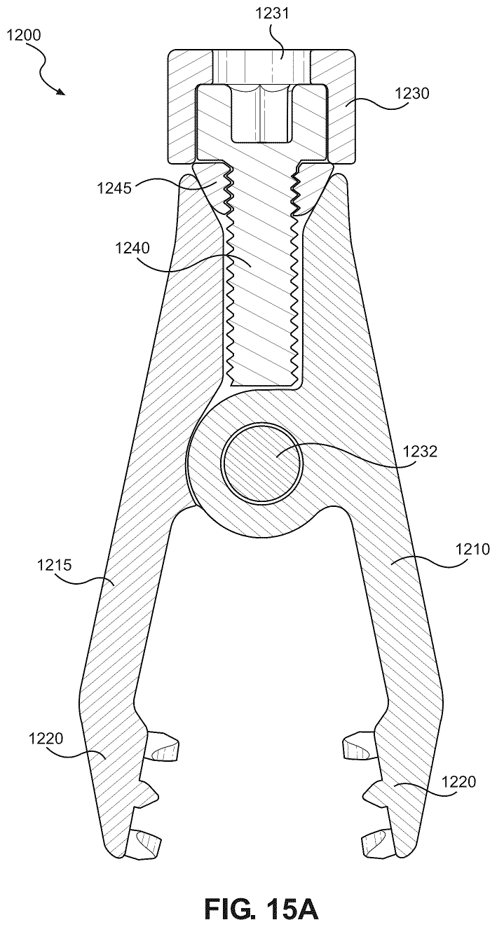

FIG. 14 illustrates in front perspective view a non-pivoting gripping component. Gripping component 1220 for surgical spinous process clamp 1200 disclosed herein can be static in some arrangements rather than pivoting as in the foregoing embodiments. As such, the gripping teeth are not able to move or pivot along the spinous process as clamping occurs. Alternatively, surgical spinous process clamp 1200 can also be implemented with pivotable gripping components as in spinous process clamp 100 above.

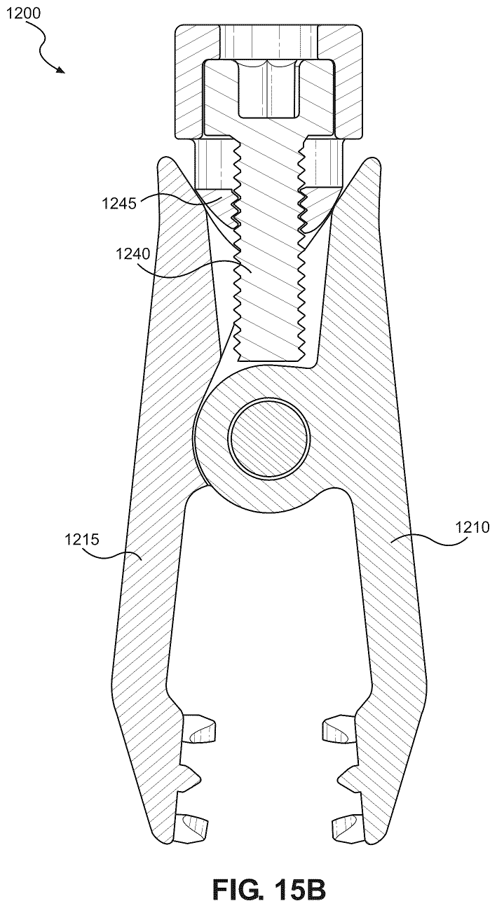

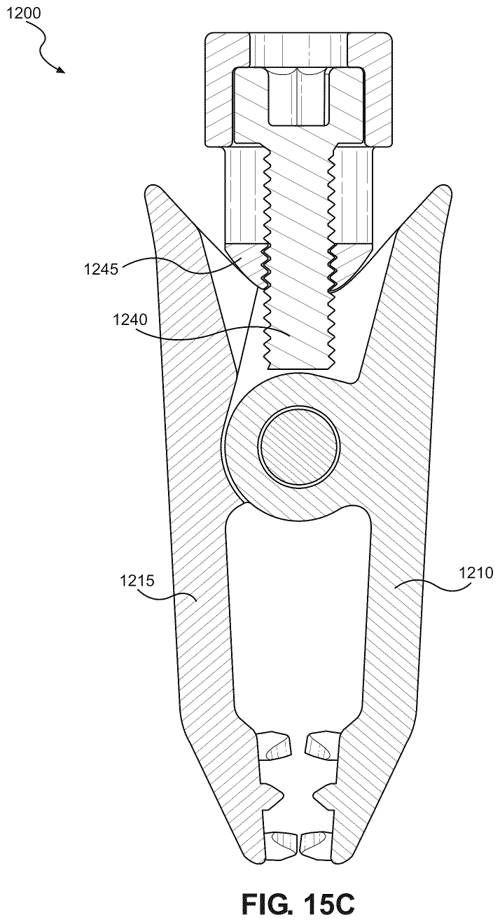

FIGS. 15A through 15C illustrate in side cross-section views an example surgical spinous process clamp in an open position, a partially closed position, and a fully closed position respectively. In some regards, the clamping mechanism of surgical spinous process clamp 1200 can closely resemble that for the clamp disclosed in in related U.S. patent application Ser. No. 18/900,106, filed Sep. 27, 2024, titled “PIVOTABLE MEDICAL DEVICE CLAMP,” which application is again hereby incorporated by reference in its entirety herein.

Threaded shaft 1240 can be rotated by way of top opening 1231 in main body 1230. This can be done by way of a hex wrench, by a coupled thumbwheel (not shown), or through any other suitable rotation means. As shaft 1240 is rotated in a clamp closing direction, traveling nut or component 1245 can be forced down the shaft and in contact with the top portions of both arms 1210, 1215. As the traveling component 1245 pushes apart the top portions of both arms 1210, 1215, the arms pivot about pin 1232 at their midsections and their bottom portions are moved closer together, which closes gripping components 1220 together to clamp onto a spinous process or anything else placed between them. This effect can be seen in surgical spinous process clamp 1200 as being open, partially closed, and fully closed across FIGS. 15A-15C.

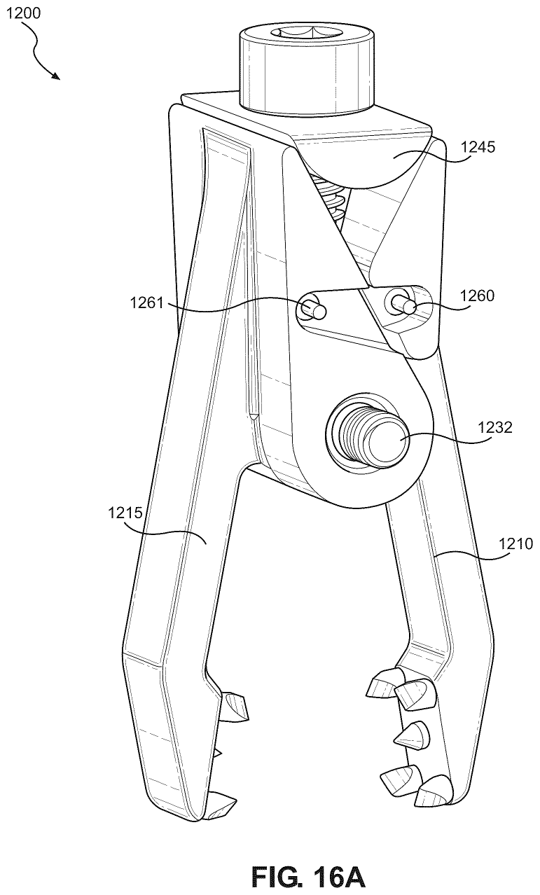

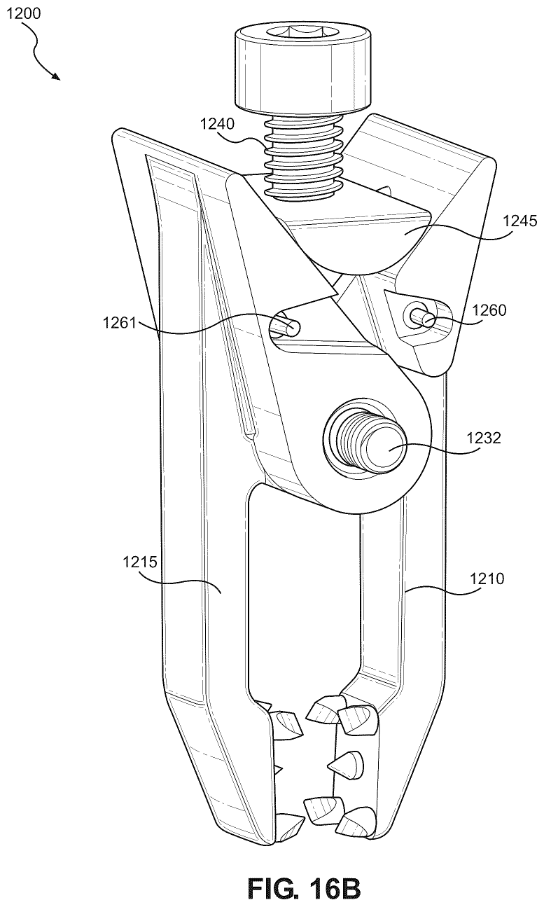

FIGS. 16A and 16B illustrate in side perspective views an example surgical spinous process clamp with its main body removed open and closed positions respectively. As can be seen with main body removed. The top portion of each arm 1210, 1215 can have a respective spring pin 1260, 1265. A spring (not shown) can be coupled to both spring pins 1260, 1265, which then results in the pins being pulled closer together. This spring bias pulling the arm top portions together then results in a default clamp position as being open, as will be readily appreciated. To close the clamp then, traveling component 1245 must be forced down threaded shaft 1240 to push against the arm top portions to counteract the spring force and push the arm top portions apart.

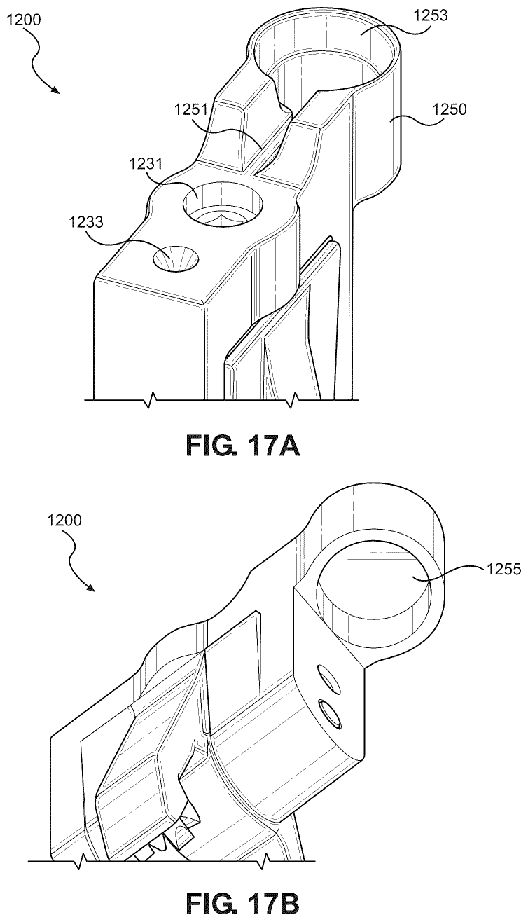

FIGS. 17A and 17B illustrate an example clamp magnetic coupling component of a surgical spinous process clamp in top perspective and bottom perspective views respectively. Clamp coupling component 1250 of surgical spinous process clamp 1200 can be configured to couple with a separate surgical device. Again, this can be a separate fiducial marker array. The coupling can be magnetic, and one or more magnets (not shown) can fit into magnet cavity 1255, which can be on the other side of opening 1253. There can be four disk magnets within magnet cavity 1255, although more or fewer magnets are also possible. Magnets can be press fit within the cavity, or a cover can be placed over the bottom opening of the cavity, as noted above.

Opening 1253 atop clamp coupling arrangement 1250 can be sized and shaped to receive a mating component from the separate surgical device. Clamp geometric features 1251 on clamp magnetic coupling arrangement 1250 can be of a specific size, shape, and geometry such that corresponding geometric features on the separate fiducial marker array or other surgical device can couple by mating specifically to be positioned and oriented in only one possible way.

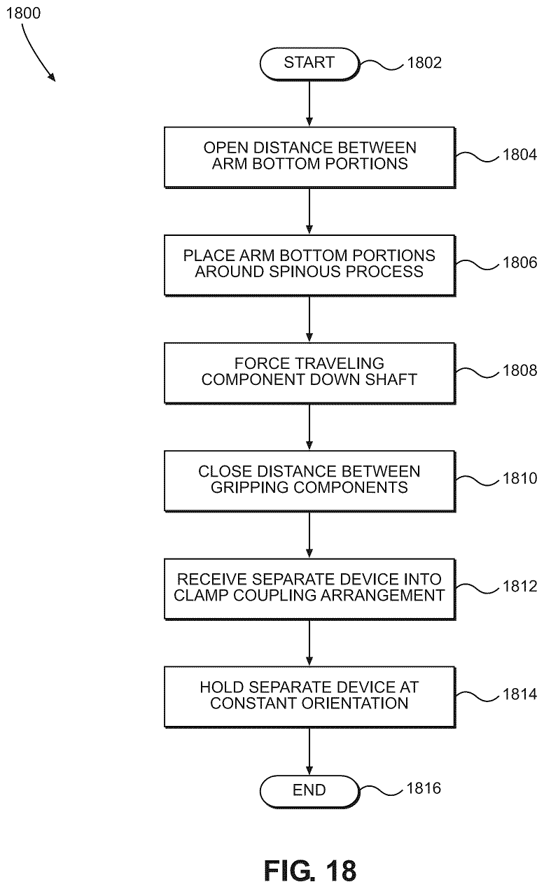

Lastly, FIG. 18 provides a flowchart of an example method of using a surgical spinous process clamp. Method 1800 can represent one possible way of using a surgical spinous process clamp, and it will be understood that various other steps, features, and details of such a detailed method are not provided here for purposes of simplicity. After a start step 1802, a first process step 1804 can involve opening a distance between bottom portions of first and second arms of the spinous process clamp. The bottom portions can each include gripping components in some arrangements. Step 1802 can be manually or automatically performed, such as where a spring within a clamp main body can be coupled to both arm top portions and configured to pull the arm top portions together, which in turn opens the arm bottom portions.

At a following process step 1806, the first and second arm bottom portions can be placed around a spinous process of a patient. This can involve placing the gripping components and the overall spinous process clamp in such a way relative to the spinous process so as to facilitate a firm gripping or clamping onto the spinous process. Step 1806 can be manually or automatically performed, such as where a separate robotic system can be configured to move and place the spinous process clamp.

At the next process step 1808, a traveling component can be forced down a shaft of the spinous process clamp so that the traveling component pushes against top portions of the first and second arms. This can be done by rotating the threaded shaft in a clamp closing direction. Step 1808 can be manually or automatically performed, such as where a separate robotic system can be configured to drive or otherwise rotate the shaft.

At subsequent process step 1810, the distance between the arm bottom portions (and thus the distance between the gripping components) of the spinous process clamp can be closed or decreased. This can be a result of the traveling component moving down the threaded shaft and against the arm top portions, which are then pushed apart by the traveling component as it moves further downward. As the arm top portions are pushed apart, the arms pivot about their midsections and their bottom portions are moved closer together. The distance between the lower portions and gripping components can accordingly be closed or decreased until they firmly grip the spinous process and affix the spinous process clamp in place relative to the patient. Step 1810 can be manually or automatically performed, such as where a separate robotic system can be configured to rotate or otherwise operate the shaft of the spinous process clamp.

The next process step 1812 can involve receiving a separate surgical device into a clamp coupling arrangement to couple a separate surgical device to the spinous process clamp. The separate surgical device can be a surgical fiducial marker positioner. The coupling can be a magnetic coupling, such as that which is set forth above, with further details being provided in related U.S. Provisional patent application Ser. No. ______/______,______, titled “MAGNETICALLY COUPLABLE FIDUCIAL MARKER ARRAYS,” which application is again hereby incorporated by reference in its entirety herein. Step 1812 can be manually or automatically performed, such as where a separate robotic system can be configured to handle and place the separate surgical device into the clamp coupling arrangement, which can be magnetic.

At the following step 1814, the separate surgical device can be set and held at a constant orientation and location with respect to the spinous process clamp while the separate surgical device remains coupled to the spinous process clamp. This can be a result of geometric features on the separate surgical device being sized and shaped so as to fit in a particular way with mating geometric features on clamp magnetic coupling arrangement. Step 1812 can be manually or automatically performed, such as where the natural physical consequence of unique mating features on both devices is to hold the separate surgical device at a particular location and orientation with respect to the clamp. The method can then end at end step 1816.

For foregoing method 1800, it will be appreciated that not all process steps are necessary, and that other process steps may be added in some arrangements. For example, changing the clamp from one spinous process to another might take place in some arrangements. Furthermore, the order of steps may be altered in some cases, and some steps may be performed simultaneously. Although known process steps are provided for the various techniques in method 1800, it will be appreciated that any other suitable similar method for using a spinous process clamp can also be used. Other variations and extrapolations of the disclosed methods will also be readily appreciated by those of skill in the art.

Although the foregoing disclosure has been described in detail by way of illustration and example for purposes of clarity and understanding, it will be recognized that the above described disclosure may be embodied in numerous other specific variations and embodiments without departing from the spirit or essential characteristics of the disclosure. Certain changes and modifications may be practiced, and it is understood that the disclosure is not to be limited by the foregoing details, but rather is to be defined by the scope of the appended claims.

What is claimed is:

1. A spinous process clamp, comprising:
a main body having a top region, a first side region, a second side region opposite the first side region, and a pin extending between the first and second side regions;
a first arm having a top portion, a bottom portion, and a midsection configured to rotate about the pin in a first rotational direction;
a second arm positioned opposite the first arm and having a top portion, a bottom portion, and a midsection configured to rotate about the pin in a second rotational direction opposite the first rotational direction;
a shaft extending through the top region of the main body;
a traveling component coupled to the shaft beneath the top region and configured to travel along the shaft and to push against the top portions of the first and second arms when the traveling component travels sufficiently downward along the shaft, wherein pushing against the top portions of the first and second arms causes the first and second arms to rotate about the pin such that the arm bottom portions clamp onto a spinous process placed therebetween; and
a clamp coupling arrangement coupled to the main body, wherein the clamp coupling arrangement is configured to couple the spinous process clamp to a separate surgical device.

2. The spinous process clamp of claim 1, further comprising:
a first gripping component located proximate the bottom portion of the first arm and configured to grip a first side of the spinous process; and
a second gripping component located proximate the bottom portion of the second arm and configured to grip a side of the spinous process opposite the first side.

3. The spinous process clamp of claim 1, wherein the separate surgical device is a fiducial marker array.

4. The spinous process clamp of claim 1, wherein the clamp coupling arrangement includes one or more clamp magnetic components configured to form a magnetic coupling to one or more separate magnetic components of the separate surgical device.

5. The spinous process clamp of claim 4, wherein the magnetic coupling results in the separate surgical device being set at a constant orientation and location with respect to the spinous process clamp while the separate surgical device remains magnetically coupled to the spinous process clamp.

6. The spinous process clamp of claim 5, wherein the shaft includes a threaded portion and the traveling component is configured to interact with the threaded portion.

7. The spinous process clamp of claim 1, further comprising:
a spring coupled to the top portion of the first arm and the top portion of the second arm, wherein the spring biases the first and second arm top portions together to open the spinous process clamp at the first and second arm bottom portions by default.

8. The spinous process clamp of claim 1, wherein the main body includes a reference divot located on a surface thereof, the reference divot being configured to facilitate calibration of another separate surgical device having a pointer tip.

9. The spinous process clamp of claim 1, wherein the shaft includes a rotational control features at the top region of the main body.

10. A method of using a spinous process clamp, the method comprising:
opening a distance between bottom portions of first and second arms of the spinous process clamp;
placing the first and second arm bottom portions around a spinous process;
forcing a traveling component down a shaft of the spinous process clamp so that the traveling component pushes against top portions of the first and second arms; and
closing the arm bottom portions onto the spinous process as the traveling component pushes against the arm top portions.

11. The method of claim 10, further comprising the steps of:
receiving a separate surgical device into the clamp coupling arrangement to couple the separate surgical device to the spinous process clamp; and
holding the separate surgical device set at a constant orientation and location with respect to the spinous process clamp while the separate surgical device remains coupled to the spinous process clamp.

12. The method of claim 11, wherein the coupling between the separate surgical device and the spinous process clamp is a magnetic coupling.
